(19) United States
(12) Patent Application Publication
(10) Pub. No.: US 2022/0054380 A1
(43) Pub. Date: Feb. 24, 2022

(54) CANNABIDIOLOIC ACID ESTERS FOR COSMETIC OR EDIBLE COMPOSITIONS

(71) Applicant: EPM (IP), Inc., San Franciso, CA (US)

(72) Inventors: Reshef Swisa, Santa Monica, CA (US); Asaf Ohana, Har Adar (IL); Salomon Sacal, Shanghai (CN)

(21) Appl. No.: 17/417,171

(22) PCT Filed: Dec. 25, 2019
(86) PCT No.: PCT/IL2019/051408

§ 371 Date: Jun. 22, 2021

Related U.S. Application Data
(60) Provisional application No. 62/784,712, filed on Dec. 25, 2018.
(60) Provisional application No. 62/784,716, filed on Dec. 25, 2018.
(60) Provisional application No. 62/863,885, filed on Jun. 20, 2019.
(60) Provisional application No. 62/863,887, filed on Jun. 20, 2019.

(51) Int. Cl. A61K 8/368; A23L 33/105; A23L 33/115; A61K 31/192; A61Q 19/08

(52) U.S. Cl. CPC A61K 8/368; A23L 33/105; A61Q 19/08; A61K 31/192; A23L 33/115

(57) ABSTRACT

The present invention provides cosmetic or edible compositions comprising a cannabinoid component, wherein the cannabinoid component comprises a cannabidiolic acid (CBDA) ester alone or in combination with one or more cannabinoid compound(s), and a physiologically acceptable carrier, excipient or diluent, and uses thereof for improving appearance, well-being and/or general health.

### FIELD OF THE INVENTION

The present invention relates to cosmetic and edible compositions comprising a cannabinoid component, wherein the cannabinoid component comprises a cannabidiolic acid (CBDA) ester alone or in combination with one or more additional cannabinoid compound(s), and a cosmetic or edible physiologically acceptable carrier, excipient or diluent, and uses thereof for improving appearance, well-being and/or general health.

### BACKGROUND OF THE INVENTION

Cannabidiol (CBD) is the major non-psychotropic phytocannabinoid compound in the plant Cannabis sativa, making up to 40% of Cannabis extracts (Grilc, Bull. Narc., 1976, 14, 37-46). It was repeatedly shown to induce anxiolytic activity in preclinical and clinical studies (Mechoulam et al., Trends Pharma. Sci., 2009, 30, 515-527; Lee et al., Br. J. Pharmacol., 2017, 174, 3242-3256). Antidepressant effects have also been reported (Alfy et al., Pharmacol. Biochem. Behav., 2010, 95, 434-442; Zanelati et al., Br. J. Pharmacol., 2010, 159, 122-128).

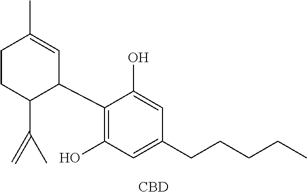

In contrast to the extensive knowledge on CBD, there is very limited literature on cannabidiolic acid (CBDA), also a major constituent of the Cannabis sativa plant, which may be due to its instability. CBDA was first isolated in 1955 (Krejčí and Šantavý, 1955, Acta Univ Palacki Olomuc 6, 59-66). Its structure was first elucidated in 1965 by analysis of the physical properties of its methyl ester, cannabidiolic acid methyl ester (CBDA-ME) (Mechoulam and Gaoni, Tetrahedron, 1965, 21, 1223-1229). Its synthesis from cannabidiol was subsequently reported (Mechoulam and Ben-Zvi, J. Chem. Soc. Commun., 1969, 7, 343-344).

The cannabinoid acids are precursors of the natural cannabinoids (Potter et al., J. Forensic Sci., 2008, 53, 90-94), potentially lowering the amount of drug required to induce effects. The decarboxylation of CBDA into CBD is enhanced by heat, indicating a relative instability of CBDA, thus lowering its potential to be a future medication (Mechoulam, Academic Press, New York, 1973, 1-99; Citti et al., J. Pharm. Biomed. Anal., 2018, 16, 532-540). Thus, CBDA-ME is a relatively unknown cannabinoid and remains understudied and its effects are only just starting to become clear.

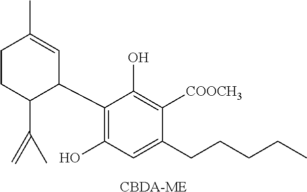

CBDA-ME is a stable derivative of CBDA and can provide pharmacological activity in vivo. Pertwee et al. (Brit. J. Pharmacology, 2018, 175, 100-112) described that the methyl ester of CBDA, designated HU-580, displays a greater potency than CBDA at suppressing signs both of acute and anticipatory nausea and of stress-induced anxiety in rats, and that it produces these effects in a 5-HT_{1A} receptor-dependent manner. Another recent study (Hen-Shoval et al., Behav. Brain Res., 2018, 351, 1-3) provides support for a potent anti-depressant effect after oral ingestion of a low dose (1 mg/kg) of HU-580 in two rat models.

WO 2018/235079 discloses compositions comprising CBDA esters and uses thereof in the treatment of a condition, disease or symptom associated with 5-HT_{1A} receptors.

U.S. Pat. No. 9,670,133 relates to compositions comprising one or multiple cannabinoid compound(s) as well as methods for their manufacture. U.S. Pat. No. 9,670,133 further discloses a composition of cannabinoids, optionally containing a minor component that may be CBDA-ME.

US Patent Application 2018/0244642 relates to compositions comprising a mixture of cannabinoid compounds and methods for their production. US Patent Application 2018/0244642 further discloses a composition of cannabinoids, optionally containing a minor component that may be cannabidivarin acid methyl ester (CBDVA-ME).

WO 2017/075374 relates to a composition of an edible base product comprising a Cannabis concentrate containing at least one cannabinoid, a starch concentrate containing tapioca maltodextrin, a rice concentrate, and a lipid concentrate containing lecithin. WO 2017/075374 further relates to a method of producing the composition comprises shearing the ingredients at a proper temperature in a dry environment.

WO 2017/053731 relates to a food additive comprising cannabinoids but lacking at least in part the taste and aroma associated with Cannabis while retaining the psychoactive and medicinal properties thereof. WO 2017/053731 further relates to a method of producing a cannabinoid-bonded polymer composition.

WO 2016/133824 relates to various cosmetics and topical formulations comprising cannabigerol, a non-psychoactive cannabinoid. The formulation may be an anti-aging cream for day or night application, a hand and nail cream, an eye cream, an acne treatment cream or tonic, an anti-diaper rash cream, a shampoo, a conditioner, a body wash, a face wash, deodorant spray, or a lip balm formulation. WO 2016/133824 further relates to methods of making the topical composition, and methods of using the topical composition for anti-aging, nourishing, cleansing, and acne treatment purposes.

There still remains an unmet need for compositions comprising non-psychoactive cannabinoid derivatives for use in non-pharmaceutical applications including in cosmetic products, foodstuffs, nutraceuticals, beverages and animal feed.

### SUMMARY OF THE INVENTION

The present invention provides cosmetic or edible compositions comprising a cannabinoid, wherein the cannabinoid component comprises a cannabidiolic acid (CBDA) ester alone or in combination with one or more additional cannabinoid compound(s), and a cosmetic or edible physiologically acceptable carrier, excipient or diluent. The compositions are useful and in improving appearance, well-being and/or general health.

It is now disclosed, unexpectedly, that the compositions comprising CBDA esters have superior effect and exhibit prolonged and significant improving effects compared to CBD or CBDA. Advantageously, the compositions disclosed herein are non-psychoactive and highly stable, thereby enabling their incorporation into a variety of non-pharmaceutical applications such as cosmetic products, foods, nutraceuticals and beverages. The compositions include any cosmetic or edible products.

According to some embodiments, the compositions for cosmetic use of the present invention achieve equal or superior cosmetic efficacy as compared to the commercially available CBD cosmetic formulations with significantly reduced skin side effects. Skin side effects may include at least one of skin irritation, skin sensitization, and chronic toxicity. The cosmetic compositions can be used in a wide range of applications such as make-up, foundation, and skin care products.

According to some embodiments, the edible or potable compositions of the present invention are useful for improving the physical well-being and/or the mental perception of well-being of a subject. In particular, the edible or potable compositions impart beneficial effects on the intestinal microbiome of the subject consuming the edible or potable composition. The edible or potable compositions can be consumed in a wide range of culinary applications such as foodstuffs and beverages, as well as in animal feed including as drinking-water additives.

It is now disclosed for the first time that the cosmetic, edible or potable composition of the present invention exhibits a strong anti-oxidative activity, and therefore it is beneficial for improving or maintaining the subject's appearance, energy levels, physical well-being and/or the mental perception of well-being.

According to one aspect, the present invention provides a cosmetic or edible composition comprising a cannabinoid component, wherein the cannabinoid component comprises a cannabidiolic acid (CBDA) ester represented by the structure of Formula (I), alone or in combination with one or more additional cannabinoid compound(s), and a physiologically acceptable carrier, excipient or diluent,

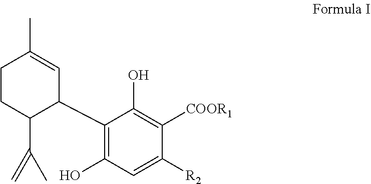

wherein
R₁ and R₂ are each independently selected from the group consisting of a linear or branched C₁-C₁₀ alkyl, a linear or branched C₂-C₁₀ alkenyl, and a linear or branched C₂-C₁₀ alkynyl; and
stereoisomers and salts thereof; and
with the proviso that when the composition comprises a CBDA ester of formula (I) wherein R₁ is methyl and R₂ is a C₃ or C₅ linear alkyl, the CBDA ester comprises more than 50% of the total weight of the cannabinoids in the composition.

According to some embodiments, R₂ is other than a C₅ linear alkyl. According to some embodiments, R₂ is other than a C₃ linear alkyl. According to some embodiments, R₁ is methyl. According to some embodiments, the cannabidiolic acid ester is cannabidiolic acid methyl ester (CBDA-ME).

According to some embodiments, the CBDA ester in the compositions of the present invention is represented by the formula (Ia):

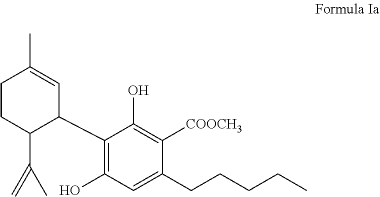 [SMILES: C=C(C)C1CCC(C)=CC1c1c(O)cc(CCCCC)c(C(=O)OC)c1O]

According to some embodiments, the CBDA ester in the compositions of the present invention is represented by the formula (Ib):

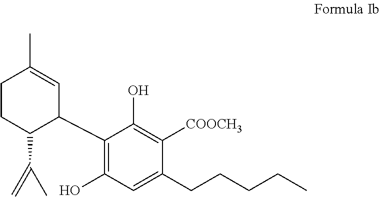

According to a particular embodiment, the CBDA ester is designated HU-580:

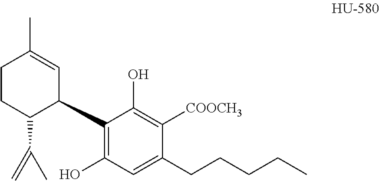

According to some embodiments, the additional cannabinoid compound is selected from the group consisting of cannabidiol (CBD), cannabigerol (CBG), Δ⁸-tetrahydrocannabinol (Δ⁸-THC), Δ⁹-tetrahydrocannabinol (Δ⁹-THC), cannabinol (CBN), Δ⁹(11)-tetrahydrocannabinol (exo-THC), cannabichromene (CBC), tetrahydrocannabinol-C3 (THC-C3), tetrahydrocannabinol-C4 (THC-C4), tetrahydrocannabinol-C7 (THC-C7), and esters and combinations thereof.

According to some embodiments the one or more additional cannabinoid compound(s) are present in one or more extracts of a Cannabis plant.

According to some embodiments, the Cannabis plant extract is obtained from a strain selected from the group consisting of Cannabis sativa, Cannabis indica, Cannabis ruderalis, a hybrid strain, and combinations thereof. According to further embodiments the Cannabis plant extract is obtained from a strain selected from the group consisting of a high-CBD strain, a high-THC strain, and a combination thereof. According to some embodiments, the Cannabis plant extract comprises at least one cannabinoid selected from the group consisting of cannabidiol (CBD), tetrahydrocannabinol (THC), cannabinol (CBN), cannabigerol (CBG), cannabichromene (CBC), acids thereof and combinations thereof.

According to some embodiments, the Cannabis plant extract comprises from about 1 to about 25% (w/w) of CBD. According to some embodiments, the Cannabis plant extract comprises about 1% (w/w) of CBD. According to some embodiments, the Cannabis plant extract comprises about 10% (w/w) of CBD. According to some embodiments, the Cannabis plant extract comprises about 25% (w/w) of CBD.

According to some embodiments, the Cannabis plant extract comprises from about 1 to about 25% (w/w) of THC. According to some embodiments, the Cannabis plant extract comprises about 1% (w/w) of THC. According to some embodiments, the Cannabis plant extract comprises about 10% (w/w) of THC. According to some embodiments, the Cannabis plant extract comprises about 25% (w/w) of THC.

According to some embodiments, the Cannabis plant extract is formed through contact with a suitable solvent or a combination of solvents. According to some embodiments, the solvent is selected from the group consisting of a polar solvent, a hydrocarbon solvent, carbon dioxide, and combinations thereof.

According to some embodiments, the composition comprises from about 0.01 to about 8% (w/w) of the cannabinoid component. According to some embodiments, the composition comprises less than about 8% (w/w) of the cannabinoid component. According to additional embodiments, the composition comprises less than about 7% (w/w) of the cannabinoid component. According to further embodiments, the composition comprises less than about 5% (w/w) of the cannabinoid component. According to yet further embodiments, composition comprises less than about 2% (w/w) of the cannabinoid component. According to still further embodiments, the composition comprises less than about 1% (w/w) of the cannabinoid component. According to additional embodiments, the composition comprises less than about 0.1% (w/w) of the cannabinoid component. According to some embodiments, the composition comprises less than about 0.01% (w/w) of the cannabinoid component.

According to some embodiments the cannabinoid component is emulsified, dissolved, dispersed or encapsulated in formulations suitable for use in either aqueous based or oil-based carriers.

According to some embodiments, the composition is a cosmetic composition selected from the group consisting of a make-up product, a foundation product, and a skin-care product. Each possibility represents a separate embodiment of the invention.

According to another aspect, the present invention provides a cosmetic composition for use in improving appearance, well-being and/or general health. According to some embodiments, the cosmetic composition comprises an excipient selected from the group consisting of viscosity modifiers, emulsifiers, film-forming agents, foaming agents, colorants, preservatives, fragrance agents, solvents, electrolytes, pH adjusting agents, and combinations thereof. Each possibility represents a separate embodiment of the invention.

According to some embodiments, the viscosity modifier is selected from the group consisting of carboxymethyl cellulose, methyl cellulose, ethyl cellulose, hydroxyethyl cellulose, hydroxypropyl cellulose, hydroxypropylmethyl cellulose, silicates, and combinations thereof.

According to some embodiments, the emulsifier is selected from the group consisting of lecithin, hydrogenated lecithin, glycerin, hydrogenated castor oil, cetyl alcohol, cetearyl alcohol, behenyl alcohol, butylene glycol, propylene glycol, and combinations thereof.

According to some embodiments, the colorant is selected from the group consisting of inorganic pigments, organic pigments, and combinations thereof. According to some embodiments, the inorganic pigment is selected from the group consisting of rutile titanium dioxide, anatase titanium dioxide, zinc oxide, zirconium oxide, iron oxides, chromium oxide, chromium hydroxide, bismuth oxy chloride, manganese violet, cerium oxide, ultramarine blue, carmine, and combinations thereof. According to some embodiments, the organic pigment is selected from the group consisting of dihydroxyacetone (DHA), erythrulose, carbonyl derivatives, and combinations thereof.

According to some embodiments, the preservative is selected from the group consisting of methyl paraben, propyl paraben, propionate salts, quaternary ammonium compounds, butyl benzoate, benzyl alcohol, benzoic acid, and combinations thereof.

According to some embodiments, the fragrance agent is selected from an essential oil obtained from a plant extract, a synthetic fragrance and combinations thereof.

According to some embodiments, the solvent is selected from a group consisting of ethanol, methanol, propanol, hexanol, propylene glycol, pentylene glycol, hexylene glycol, 1, 2-octanediol, sorbitol, glycerol, ethyl acetate, and combinations thereof.

According to some embodiments, the electrolyte is sodium chloride.

According to some embodiments, the pH adjusting agent is selected from the group consisting of organic and mineral acids, or bases.

According to some embodiments, the cosmetic composition further comprises a cosmetically-active ingredient selected from the group consisting of sunscreen agents, anti-wrinkle and anti-aging agents, skin peel agents, whitening and bleaching agents, sunless tanning agents, vitamins, skin conditioning agents, and combinations thereof.

According to some embodiments, the cosmetic composition is in the form selected from the group consisting of powder, ointment, cream, milk, lotion, gel, emulsion, emulsified gel, mousse, foam, spray, liquid, paste, serum, and aerosol.

According to another aspect, the present invention provides a method for improving the appearance of skin comprising applying topically to the skin of a subject the cosmetic composition as described herein.

According to some embodiments, the composition is an edible composition selected from the group consisting of food products, nutraceuticals, beverages, and animal feed.

According to another aspect, the present invention provides an edible composition is comprising compound of formula I as described hereinabove for use in improving appearance, well-being and/or general health.

According to some embodiments, the edible composition comprises a phospholipid selected from the group consisting of soy lecithin, egg lecithin, hydrogenated soy lecithin, hydrogenated egg lecithin, and a combination thereof. According to some embodiments, the phospholipid forms micelles, emulsions or liposomes. According to some embodiments, the phospholipid forms liposomes. According to some embodiments, the composition further comprising cholesterol.

According to some embodiments, the edible composition further comprises an edible excipient selected from the group consisting of fillers, emulsifiers, anticaking agents, preservatives, colorants, flavoring agents, and combinations thereof.

According to some embodiments, the emulsifier is selected from the group consisting of hydrogenated lecithin, sodium phosphates, mono- and diglycerides, sodium stearoyl lactylate, diacetyl tartaric acid ester of monoglyceride, and combinations thereof.

According to some embodiments, the anticaking agent is selected from the group consisting of cellulose, microcrystalline cellulose, silicon dioxide, tri-calcium phosphate, sodium chloride, sodium bicarbonate, sodium aluminum silicate, magnesium stearate, magnesium carbonate, and combinations thereof.

According to some embodiments, the preservative is selected from the group consisting of sulfur dioxide and sulfites, sorbic acid, sodium sorbate, calcium sorbate, potassium sorbate, benzoic acid, sodium benzoate, potassium benzoate, lactic acid, propionic acid, sodium propionate, and combinations thereof. According to some embodiments, the colorant is selected from the group consisting of brilliant blue (E133), indigotine (E132), fast green (E143), erythrosine (E127), allura red (E129), tartrazine (E102), sunset yellow (E110), and natural food dyes. According to some embodiments, the natural food dye is selected from the group consisting of annatto (E160b), caramel (E150a-d), carmine (E120), dactylopius coccus, elderberry juice (E163), lycopene (E160d), paprika (E160c), turmeric (E100), and combinations thereof.

According to some embodiments, the flavoring agent is extracted from apple, cherry, green tea, cinnamon, clove, black tea, plum, mango, date, watermelon, coconut, pear, jasmine, peach, fennel, melon, lychee, mint, chocolate, coffee, cream, banana, almond, grape, strawberry, blueberry, blackberry, pine, kiwi, sapote, taro, lotus, pineapple, orange, lemon, licorice, vanilla, rose, osmanthus, ginseng, spearmint, citrus, cucumber, honeydew, walnut, honey, and combinations thereof.

According to some embodiments, the edible composition further comprises a nutrient selected from the group consisting of vitamins, minerals, and combinations thereof.

According to some embodiments, the vitamin is selected from the group consisting of vitamin A (retinols and carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), vitamin K (quinones), and combinations thereof.

According to some embodiments, the mineral is selected from the group consisting of magnesium, calcium, iron, zinc, chrome, selenium, potassium, silicon, and combinations thereof.

According to some embodiments, the edible composition is in a form selected from the group consisting of powder, flake, granular, capsule, tablet, syrup, solution, emulsion and suspension. In some embodiments, the food product is selected from bread, cereals, pasta, pastry, biscuits, candy, and confectionary. In some embodiments, the edible composition is a beverage.

According to another aspect, the present invention provides a method for improving physical well-being and/or the mental perception of well-being of a subject comprising consuming by said subject the edible composition as described hereinabove.

### DETAILED DESCRIPTION OF THE INVENTION

The present invention is directed to cosmetic and edible compositions comprising a cannabinoid component, wherein the cannabinoid component comprises a cannabidiolic acid (CBDA) ester alone or in combination with one or more additional cannabinoid compound(s), and a physiologically acceptable carrier, excipient or diluent, and uses thereof for improving appearance, well-being and/or general health. Optionally, in some embodiments, the one or more additional cannabinoid compound(s) is present in extracts of a Cannabis plant.

According to one aspect, the present invention provides a cosmetic or edible composition comprising a cannabinoid component, wherein the cannabinoid component comprises a cannabidiolic acid (CBDA) ester represented by the structure of Formula (I), alone or in combination with one or more additional cannabinoid compound(s), and a physiologically acceptable carrier, excipient or diluent,

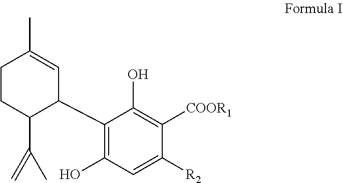

wherein
R₁ and R₂ are each independently selected from the group consisting of a linear or branched C₁-C₁₀ alkyl, a linear or branched C₂-C₁₀ alkenyl, and a linear or branched C₂-C₁₀ alkynyl; and
stereoisomers and salts thereof; and
with the proviso that when the composition comprises a CBDA ester of formula (I) wherein R₁ is methyl and R₂ is C₃ or C₅ linear alkyl, the CBDA ester comprises more than 50% of the total weight of the cannabinoids in the composition.

According to another aspect, the present invention provides a cosmetic or edible composition comprising a cannabinoid component, wherein the cannabinoid component comprises a cannabidiolic acid (CBDA) ester represented by the structure of Formula (I), alone or in combination with one or more additional cannabinoid compound(s), and a physiologically acceptable carrier, excipient or diluent,

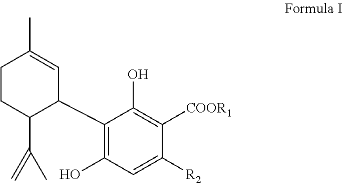

wherein
R₁ and R₂ are each independently selected from the group consisting of a linear or branched C₁-C₁₀ alkyl, a linear or branched C₂-C₁₀ alkenyl, and a linear or branched C₂-C₁₀ alkynyl; and
stereoisomers and salts thereof; and
with the proviso that when the composition comprises a CBDA ester of formula (I) wherein R₁ is methyl and R₂ is C₃ or C₅ linear alkyl, the CBDA ester comprises more than 51% of the total weight of the cannabinoids in the composition.

According to another aspect, the present invention provides a cosmetic or edible composition comprising a cannabinoid component, wherein the cannabinoid component comprises a cannabidiolic acid (CBDA) ester represented by the structure of Formula (I), alone or in combination with one or more additional cannabinoid compound(s), and a physiologically acceptable carrier, excipient or diluent,

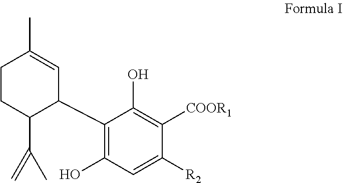

wherein
R₁ and R₂ are each independently selected from the group consisting of a linear or branched C₁-C₁₀ alkyl unsubstituted or substituted by one or more groups selected from the group consisting of hydroxyl, halogen, amino, thiol, and phosphate, a linear or branched C₂-C₁₀ alkenyl unsubstituted or substituted by one or more groups selected from the group consisting of hydroxyl, halogen, amino, thiol, and phosphate, and a linear or branched C₂-C₁₀ alkynyl unsubstituted or substituted by one or more groups selected from the group consisting of hydroxyl, halogen, amino, thiol, and phosphate; and
stereoisomers and salts thereof; and
with the proviso that when the composition comprises a CBDA ester of formula (I) wherein R₁ is methyl and R₂ is C₃ or C₅ linear alkyl, the CBDA ester comprises more than 50% of the total weight of the cannabinoids in the composition.

### Cannabinoid Component

The word “cannabinoid” as used herein refers to any compound that interacts with cannabinoid receptors including endocannabinoids (produced naturally in the body by humans and animals), phytocannabinoids (found in Cannabis and some other plants), and synthetic cannabinoids (manufactured artificially). The term “cannabinoid acid” refers to the acid form of the above-mentioned cannabinoids.

The word “cannabidiol” refers to cannabidiol (CBD) and CBD derivatives. CBD may be obtained from industrial hemp extract with a trace amount of THC or from Cannabis extract using high CBD Cannabis cultivars. According to some embodiments, cannabidiol may be obtained from plant extract, or may be prepared synthetically (manufactured artificially).

The abbreviation “CBDA” is used herein to refer to cannabidiolic acid, which is the acid form of CBD. The term “cannabidiolic acid ester” or “cannabidiolic ester” refers to various molecules, which are the alkyl, alkenyl, alkynyl or aryl ester form of CBDA. The abbreviation “CBDA-ME” is used herein to refer to cannabidiolic acid methyl ester, which is the methyl ester form of CBDA.

It is to be understood that the compounds provided herein may contain one or more chiral centers. Such chiral centers may each be of either of the (R) or (S) configuration. In case a compound of the invention contains more than one chiral center, each one of those chiral centers may be of the (R) or (S) configuration, independently. Thus, the compounds provided herein may be enantiomerically pure, or be stereoisomeric or diastereomeric mixtures.

According to some embodiments, R₁ is a linear or branched C₁-C₁₀ alkyl. According to some embodiments, R₁ is a linear or branched C₂-C₁₀ alkenyl. According to some embodiments, R₁ is a linear or branched C₂-C₁₀ alkynyl.

According to some embodiments, R₁ is selected from the group consisting of a linear or branched C₁-C₅ unsubstituted alkyl, a linear or branched C₇-C₁₀ unsubstituted alkyl, a linear or branched C₁-C₁₀ halo alkyl, a linear or branched C₂-C₁₀ alkenyl, and a linear or branched C₂-C₁₀ alkynyl. According to some embodiments, R₁ is a linear or branched C₁-C₅ unsubstituted alkyl. According to some embodiments, R₁ is a linear or branched C₇-C₁₀ unsubstituted alkyl. According to some embodiments, R₁ is a linear or branched C₁-C₁₀ halo alkyl. Each possibility represents a separate embodiment of the present invention.

According to some embodiments, R₁ is methyl. According to some embodiments, the cannabidiolic acid ester is cannabidiolic acid methyl ester (CBDA-ME).

According to some embodiments, R₂ is a linear or branched C₁-C₁₀ alkyl. According to some embodiments, R₂ is a linear or branched C₂-C₁₀ alkenyl. According to some embodiments, R₂ is a linear or branched C₂-C₁₀ alkynyl. According to some embodiments, R₂ is a linear C₅H₁₁.

According to some embodiments, R₂ is selected from the group consisting of a C₁ or C₂ alkyl, a linear or branched C₄ alkyl, a linear or branched C₆-C₁₀ alkyl, a linear or branched C₂-C₁₀ alkenyl, and a linear or branched C₂-C₁₀ alkynyl.

According to some embodiments, R₂ is methyl. According to some embodiments, R₂ is ethyl. According to some embodiments, R₂ is a linear or branched C₃ alkyl. According to some embodiments, R₂ is a linear or branched C₄ alkyl. According to some embodiments, R₂ is a linear or branched C₅ alkyl. According to some embodiments, R₂ is a linear or branched C₆-C₁₀ alkyl. According to some embodiments, R₂ is other than C₅ linear alkyl. According to some embodiments, R₂ is other than a C₃ linear alkyl. Each possibility represents a separate embodiment of the present invention.

According to some embodiments, R₁ is a linear or branched C₁-C₁₀ alkyl and R₂ is a linear or branched C₁-C₁₀ alkyl. According to some embodiments, R₁ is a linear or branched C₁-C₁₀ alkyl and R₂ is a linear or branched C₂-C₁₀ alkenyl. According to some embodiments, R₁ is a linear or branched C₁-C₁₀ alkyl and R₂ is a linear or branched C₂-C₁₀ alkynyl. According to some embodiments, R₁ is a linear or branched C₁-C₁₀ alkyl and R₂ is a linear C₃H₇. According to some embodiments, R₁ is a linear or branched C₁-C₁₀ alkyl and R₂ is a linear C₅H₁₁. Each possibility represents a separate embodiment. According to some embodiments, R₁ is a linear or branched C₁-C₁₀ alkyl and R₂ is methyl. According to some embodiments, R₁ is a linear or branched C₁-C₁₀ alkyl and R₂ is ethyl. According to some embodiments, R₁ is a linear or branched C₁-C₁₀ alkyl and R₂ is a linear or branched C₃ alkyl. According to some embodiments, R₁ is a linear or branched C₁-C₁₀ alkyl and R₂ is a branched C₃ alkyl. According to some embodiments, R₁ is a linear or branched C₁-C₁₀ alkyl and R₂ is a linear or branched C₄ alkyl. According to some embodiments, R₁ is a linear or branched C₁-C₁₀ alkyl and R₂ is a linear or branched C₅ alkyl. According to some embodiments, R₁ is a linear or branched C₁-C₁₀ alkyl and R₂ is a branched C₅ alkyl. According to some embodiments, R₁ is a linear or branched C₁-C₁₀ alkyl and R₂ is a linear or branched C₆-C₁₀ alkyl. According to some embodiments, R₁ is a linear or branched C₁-C₁₀ alkyl and R₂ is other than C₅ linear alkyl. According to some embodiments, R₁ is a linear or branched C₁-C₁₀ alkyl and R₂ is other than a C₃ linear alkyl. Each possibility represents a separate embodiment.

According to some embodiments, R₁ is a linear or branched C₂-C₁₀ alkenyl and R₂ is a linear or branched C₁-C₁₀ alkyl. According to some embodiments, R₁ is a linear or branched C₂-C₁₀ alkenyl and R₂ is a linear or branched C₂-C₁₀ alkenyl. According to some embodiments, R₁ is a linear or branched C₂-C₁₀ alkenyl and R₂ is a linear or branched C₂-C₁₀ alkynyl. According to some embodiments, R₁ is a linear or branched C₂-C₁₀ alkenyl and R₂ is a linear C₃H₇. According to some embodiments, R₁ is a linear or branched C₂-C₁₀ alkenyl and R₂ is a linear C₅H₁₁. Each possibility represents a separate embodiment. According to some embodiments, R₁ is a linear or branched C₂-C₁₀ alkenyl and R₂ is methyl. According to some embodiments, R₁ is a linear or branched C₂-C₁₀ alkenyl and R₂ is ethyl. According to some embodiments, R₁ is a linear or branched C₂-C₁₀ alkenyl and R₂ is a linear or branched C₃ alkyl. According to some embodiments, R₁ is a linear or branched C₂-C₁₀ alkenyl and R₂ is a branched C₃ alkyl. According to some embodiments, R₁ is a linear or branched C₂-C₁₀ alkenyl and R₂ is a linear or branched C₄ alkyl. According to some embodiments, R₁ is a linear or branched C₂-C₁₀ alkenyl and R₂ is a linear or branched C₅ alkyl. According to some embodiments, R₁ is a linear or branched C₂-C₁₀ alkenyl and R₂ is a branched C₅ alkyl. According to some embodiments, R₁ is a linear or branched C₂-C₁₀ alkenyl and R₂ is a linear or branched C₆-C₁₀ alkyl. According to some embodiments, R₁ is a linear or branched C₂-C₁₀ alkenyl and R₂ is other than C₅ linear alkyl. According to some embodiments, R₁ is a linear or branched C₂-C₁₀ alkenyl and R₂ is other than a C₃ linear alkyl. Each possibility represents a separate embodiment.

According to some embodiments, R₁ is a linear or branched C₂-C₁₀ alkynyl and R₂ is a linear or branched C₁-C₁₀ alkyl. According to some embodiments, R₁ is a linear or branched C₂-C₁₀ alkynyl and R₂ is a linear or branched C₂-C₁₀ alkenyl. According to some embodiments, R₁ is a linear or branched C₂-C₁₀ alkynyl and R₂ is a linear or branched C₂-C₁₀ alkynyl. According to some embodiments, R₁ is a linear or branched C₂-C₁₀ alkynyl and R₂ is a linear C₃H₇. According to some embodiments, R₁ is a linear or branched C₂-C₁₀ alkynyl and R₂ is a linear C₅H₁₁. Each possibility represents a separate embodiment. According to some embodiments, R₁ is a linear or branched C₂-C₁₀ alkynyl and R₂ is methyl. According to some embodiments, R₁ is a linear or branched C₂-C₁₀ alkynyl and R₂ is ethyl. According to some embodiments, R₁ is a linear or branched C₂-C₁₀ alkynyl and R₂ is a linear or branched C₃ alkyl. According to some embodiments, R₁ is a linear or branched C₂-C₁₀ alkynyl and R₂ is a branched C₃ alkyl. According to some embodiments, R₁ is a linear or branched C₂-C₁₀ alkynyl and R₂ is a linear or branched C₄ alkyl. According to some embodiments, R₁ is a linear or branched C₂-C₁₀ alkynyl and R₂ is a linear or branched C₅ alkyl. According to some embodiments, R₁ is a linear or branched C₂-C₁₀ alkynyl and R₂ is a branched C₅ alkyl. According to some embodiments, R₁ is a linear or branched C₂-C₁₀ alkynyl and R₂ is a linear or branched C₆-C₁₀ alkyl. According to some embodiments, R₁ is a linear or branched C₂-C₁₀ alkynyl and R₂ is other than C₅ linear alkyl. According to some embodiments, R₁ is a linear or branched C₂-C₁₀ alkynyl and R₂ is other than a C₃ linear alkyl. Each possibility represents a separate embodiment.

According to some embodiments, R₁ is a linear or branched C₁-C₅ unsubstituted alkyl and R₂ is a linear or branched C₁-C₁₀ alkyl. According to some embodiments, R₁ is a linear or branched C₁-C₅ unsubstituted alkyl and R₂ is a linear or branched C₂-C₁₀ alkenyl. According to some embodiments, R₁ is a linear or branched C₁-C₅ unsubstituted alkyl and R₂ is a linear or branched C₂-C₁₀ alkynyl. According to some embodiments, R₁ is a linear or branched C₁-C₅ unsubstituted alkyl and R₂ is a linear C₃H₇. According to some embodiments, R₁ is a linear or branched C₁-C₅ unsubstituted alkyl and R₂ is a linear C₅H₁₁. Each possibility represents a separate embodiment. According to some embodiments, R₁ is a linear or branched C₁-C₅ unsubstituted alkyl and R₂ is methyl. According to some embodiments, R₁ is a linear or branched C₁-C₅ unsubstituted alkyl and R₂ is ethyl. According to some embodiments, R₁ is a linear or branched C₁-C₅ unsubstituted alkyl and R₂ is a linear or branched C₄ alkyl. According to some embodiments, R₁ is a linear or branched C₁-C₅ unsubstituted alkyl and R₂ is a linear or branched C₃ alkyl. According to some embodiments, R₁ is a linear or branched C₁-C₅ unsubstituted alkyl and R₂ is a branched C₃ alkyl. According to some embodiments, R₁ is a linear or branched C₁-C₅ unsubstituted alkyl and R₂ is a linear or branched C₄ alkyl. According to some embodiments, R₁ is a linear or branched C₁-C₅ unsubstituted alkyl and R₂ is a branched C₅ alkyl. According to some embodiments, R₁ is a linear or branched C₁-C₅ unsubstituted alkyl and R₂ is a linear or branched C₅ alkyl. According to some embodiments, R₁ is a linear or branched C₁-C₅ unsubstituted alkyl and R₂ is a linear or branched C₆-C₁₀ alkyl. According to some embodiments, R₁ is a linear or branched C₁-C₅ unsubstituted alkyl and R₂ is other than C₅ linear alkyl. According to some embodiments, R₁ is a linear or branched C₁-C₅ unsubstituted alkyl and R₂ is other than a C₃ linear alkyl. Each possibility represents a separate embodiment.

According to some embodiments, R₁ is a linear or branched C₇-C₁₀ unsubstituted alkyl and R₂ is a linear or branched C₁-C₁₀ alkyl. According to some embodiments, R₁ is a linear or branched C₇-C₁₀ unsubstituted alkyl and R₂ a linear or branched C₂-C₁₀ alkenyl. According to some embodiments, R₁ is a linear or branched C₇-C₁₀ unsubstituted alkyl and R₂ is a linear or branched C₂-C₁₀ alkynyl. According to some embodiments, R₁ is a linear or branched C₇-C₁₀ unsubstituted alkyl and R₂ a linear C₃H₇. According to some embodiments, R₁ is a linear or branched C₇-C₁₀ unsubstituted alkyl and R₂ a linear C₅H₁₁. Each possibility represents a separate embodiment. According to some embodiments, R₁ is a linear or branched C₇-C₁₀ unsubstituted alkyl and R₂ is methyl. According to some embodiments, R₁ is a linear or branched C₇-C₁₀ unsubstituted alkyl and R₂ is ethyl. According to some embodiments, R₁ is a linear or branched C₇-C₁₀ unsubstituted alkyl and R₂ is a linear or branched C₃ alkyl. According to some embodiments, R₁ is a linear or branched C₇-C₁₀ unsubstituted alkyl and R₂ is a branched C₃ alkyl. According to some embodiments, R₁ is a linear or branched C₇-C₁₀ unsubstituted alkyl and R₂ is a linear or branched C₄ alkyl. According to some embodiments, R₁ is a linear or branched C₇-C₁₀ unsubstituted alkyl and R₂ is a linear or branched C₅ alkyl. According to some embodiments, R₁ is a linear or branched C₇-C₁₀ unsubstituted alkyl and R₂ is a branched C₅ alkyl. According to some embodiments, R₁ is a linear or branched C₇-C₁₀ unsubstituted alkyl and R₂ is a linear or branched C₆-C₁₀ alkyl. According to some embodiments, R₁ is a linear or branched C₇-C₁₀ unsubstituted alkyl and R₂ is other than C₅ linear alkyl. According to some embodiments, R₁ is a linear or branched C₇-C₁₀ unsubstituted alkyl and R₂ is other than a C₃ linear alkyl. Each possibility represents a separate embodiment.

According to some embodiments, R₁ is a linear or branched C₁-C₁₀ halo alkyl and R₂ is a linear or branched C₁-C₁₀ alkyl. According to some embodiments, R₁ is a linear or branched C₁-C₁₀ halo alkyl and R₂ a linear or branched C₂-C₁₀ alkenyl. According to some embodiments, R₁ is a linear or branched C₁-C₁₀ halo alkyl and R₂ is a linear or branched C₂-C₁₀ alkynyl. According to some embodiments, R₁ is a linear or branched C₁-C₁₀ halo alkyl and R₂ a linear C₃H₇. According to some embodiments, R₁ is a linear or branched C₁-C₁₀ halo alkyl and R₂ a linear C₅H₁₁. Each possibility represents a separate embodiment. According to some embodiments, R₁ is a linear or branched C₁-C₁₀ halo alkyl and R₂ is methyl. According to some embodiments, R₁ is a linear or branched C₁-C₁₀ halo alkyl and R₂ is ethyl. According to some embodiments, R₁ is a linear or branched C₁-C₁₀ halo alkyl and R₂ is a linear or branched C₃ alkyl. According to some embodiments, R₁ is a linear or branched C₁-C₁₀ halo alkyl and R₂ is a branched C₃ alkyl. According to some embodiments, R₁ is a linear or branched C₁-C₁₀ halo alkyl and R₂ is a linear or branched C₄ alkyl. According to some embodiments, R₁ is a linear or branched C₁-C₁₀ halo alkyl and R₂ is a linear or branched C₅ alkyl. According to some embodiments, R₁ is a linear or branched C₁-C₁₀ halo alkyl and R₂ is a branched C₅ alkyl. According to some embodiments, R₁ is a linear or branched C₁-C₁₀ halo alkyl and R₂ is a linear or branched C₆-C₁₀ alkyl. According to some embodiments, R₁ is a linear or branched C₁-C₁₀ halo alkyl and R₂ is other than C₅ linear alkyl. According to some embodiments, R₁ is a linear or branched C₁-C₁₀ halo alkyl and R₂ is other than a C₃ linear alkyl. Each possibility represents a separate embodiment.

According to some embodiments, R₁ is methyl and R₂ is a linear or branched C₁-C₁₀ alkyl. According to some embodiments, R₁ is methyl and R₂ a linear or branched C₂-C₁₀ alkenyl. According to some embodiments, R₁ is methyl and R₂ is a linear or branched C₂-C₁₀ alkynyl. According to some embodiments, R₁ is methyl and R₂ a linear C₃H₇. According to some embodiments, R₁ is methyl and R₂ a linear C₅H₁₁. Each possibility represents a separate embodiment. According to some embodiments, R₁ is methyl and R₂ is methyl. According to some embodiments, R₁ is methyl and R₂ is ethyl. According to some embodiments, R₁ is methyl and R₂ is a linear or branched C₃ alkyl. According to some embodiments, R₁ is methyl and R₂ is a branched C₃ alkyl. According to some embodiments, R₁ is methyl and R₂ is a linear or branched C₄ alkyl. According to some embodiments, R₁ is methyl and R₂ is a linear or branched C₅ alkyl. According to some embodiments, R₁ is methyl and R₂ is a branched C₅ alkyl. According to some embodiments, R₁ is methyl and R₂ is a linear or branched C₆-C₁₀ alkyl. According to some embodiments, R₁ is methyl and R₂ is other than C₅ linear alkyl. According to some embodiments, R₁ is methyl and R₂ is other than a C₃ linear alkyl. Each possibility represents a separate embodiment.

According to some embodiments, the CBDA ester of the present invention is represented by the formula (Ia):

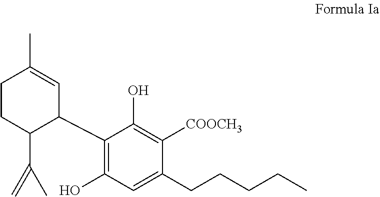 [SMILES: C=C(C)C1CCC(C)=CC1c1c(O)cc(CCCCC)c(C(=O)OC)c1O]

According to some embodiments, the CBDA ester of the present invention is represented by the formula (Ib):

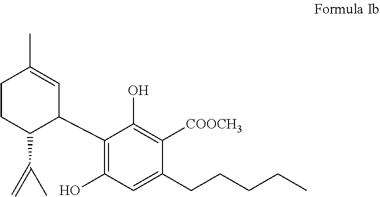

According to a particular embodiment, the CBDA ester is designated HU-580:

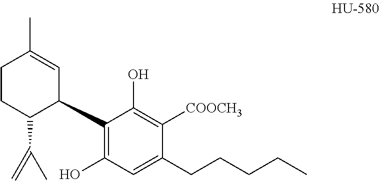

An “alkyl” group refers to a saturated aliphatic hydrocarbon, including straight-chain or linear-chain, branched-chain and cycloalkyl groups. In one embodiment, the alkyl group has 1-10 carbons designated here as C₁-C₁₀-alkyl. In another embodiment, the alkyl group has 2-5 carbons designated here as C₁-C₅-alkyl. In another embodiment, the alkyl group has 2-4 carbons designated here as C₂-C₄-alkyl. Each possibility represents a separate embodiment of the invention. The alkyl group may be unsubstituted or substituted by one or more groups selected from the group consisting of hydroxyl, halogen, amino, thiol, phosphate, and combinations thereof.

A “cycloalkyl” group refers to a non-aromatic mono- or multicyclic ring system. In one embodiment, the cycloalkyl group has 3-10 carbon atoms. In another embodiment, the cycloalkyl group has 5-10 carbon atoms. Exemplary monocyclic cycloalkyl groups include cyclopentyl, cyclohexyl, cycloheptyl and the like. An alkylcycloalkyl is an alkyl group as defined herein bonded to a cycloalkyl group as defined herein. The cycloalkyl group can be unsubstituted or substituted with any one or more of the substituents defined above for alkyl.

The terms “halo” and “halogen” refer to the fluoro, chloro, bromo or iodo atoms. There can be one or more halogens, which are the same or different.

An “alkenyl” group refers to an aliphatic hydrocarbon group containing at least one carbon-carbon double bond including straight-chain, linear-chain, branched-chain and cyclic alkenyl groups. In one embodiment, the alkenyl group has 2-10 carbon atoms (a C₂₋₁₀ alkenyl). In another embodiment, the alkenyl group has 2-4 carbon atoms in the chain (a C₂₋₄ alkenyl). Exemplary alkenyl groups include, but are not limited to, ethenyl, propenyl, n-butenyl, i-butenyl, 3-methylbut-2-enyl, n-pentenyl, heptenyl, octenyl, cyclohexyl-butenyl and decenyl. An alkylalkenyl is an alkyl group as defined herein bonded to an alkenyl group as defined herein. The alkenyl group can be unsubstituted or substituted through available carbon atoms with one or more groups defined hereinabove for alkyl.

An “alkynyl” group refers to an aliphatic hydrocarbon group containing at least one carbon-carbon triple bond including straight-chain or linear-chain and branched-chain. In one embodiment, the alkynyl group has 2-10 carbon atoms in the chain (a C₂₋₁₀ alkynyl). In another embodiment, the alkynyl group has 2-4 carbon atoms in the chain (a C₂₋₄ alkynyl). Exemplary alkynyl groups include, but are not limited to, ethynyl, propynyl, n-butynyl, 2-butynyl, 3-methylbutynyl, n-pentynyl, heptynyl, octynyl and decynyl. An alkylalkynyl is an alkyl group as defined herein bonded to an alkynyl group as defined herein. The alkynyl group can be unsubstituted or substituted through available carbon atoms with one or more groups defined hereinabove for alkyl.

According to some embodiments, the additional cannabinoid compound is selected from the group consisting of cannabidiol (CBD), cannabigerol (CBG), Δ⁸-tetrahydrocannabinol (Δ⁸-THC), Δ⁹-tetrahydrocannabinol (Δ⁹-THC), cannabinol (CBN), Δ⁹(11)-tetrahydrocannabinol (exo-THC), cannabichromene (CBC), tetrahydrocannabinol-C3 (THC-C3), tetrahydrocannabinol-C4 (THC-C4), tetrahydrocannabinol-C7 (THC-C7), esters thereof and combinations thereof.

In some embodiments, the CBDA ester of Formula (I) or the cannabinoid compound includes a solvate. The term “solvate” as used herein refers to a physical association of a compound disclosed herein with one or more solvent molecules. This physical association involves varying degrees of ionic and covalent bonding, including hydrogen bonding. In certain instances, the solvate will be capable of isolation. “Solvate” encompasses both solution-phase and isolatable solvates. Non-limiting examples of suitable solvates include ethanolates, methanolates and the like. A “hydrate” is a solvate in which the solvent molecule is water.

In embodiments in which the CBDA ester is incorporated into a composition in solid state form, the present disclosure also includes any polymorphs thereof. The term “polymorph” refers to a particular crystalline or amorphous state of a substance, which can be characterized by particular physical properties such as X-ray diffraction, electron diffraction, IR spectra, Raman spectra, melting point, and the like.

Any of the cannabinoids disclosed herein, specifically, the CBDA ester of Formula (I), can be prepared by any manner known to those skilled in the art. For example, it may be isolated or extracted from a natural source or prepared by synthetic or semi-synthetic means. For example, cannabinoids can be isolated by extraction from Cannabis plants. Plants in the Cannabis genus include, but are not limited to, Cannabis sativa, Cannabis indica, and Cannabis ruderalis. Each possibility represents a separate embodiment. These plants are the natural sources of cannabinoids. According to some embodiments, certain cannabinoids are isolated or extracted from Cannabis plants and then derivatized to the CBDA ester of Formula (I).

The term “extract” as used herein refers a product prepared by extraction by physical means (e.g. by comminuting, pressing, heating, pulsed electric field assisted treatments, shear treatments and pressure wave treatments), by chemical means (e.g. by treatment with an acid, a base, a solvent) and/or by biochemical means (e.g. by treatment with hydrolytic enzymes, microorganisms). The term refers to a liquid substance obtained through extraction from a given substance, or to a concentrate or essence which is free of, or substantially free of solvent. The term extract may be a single extract obtained from a particular extraction step or series of extraction steps. Extract also may be a combination of extracts obtained from separate extraction steps or separate feedstocks. Such combined extracts are thus also encompassed by the term “extract”. Any methods of extraction with suitable solvent are encompassed. Exemplary extraction methods can be found for example in U.S. Pat. No. 6,403,126, the contents of which are incorporated by reference herein. The extract may be obtained from any part of the plant e.g. from leaves, flowers, stems, roots, fruits and seeds. The extract may be aqueous or oily.

As used herein, the term “non-pharmaceutical”, when referring herein to non-pharmaceutical uses and compositions, means uses other than those that provide medicaments to a person for the purpose of treating diseases or other medical disorder. The composition includes any cosmetic or edible compositions.

The term “physiologically acceptable carrier, excipient or diluent” is intended to mean, a carrier that is generally recognized as safe for consumption by a human or non-human animal or for use on the skin or hair of the animal More specifically the acceptable carrier, excipient or diluent is suitable for use in contact with the cells of humans and animals without toxicity, irritation, allergic response, and the like. The physiologically acceptable carrier, excipient or diluent includes but not limited to a cosmetically acceptable carrier or an edible or potable carrier. It is appreciated that all carriers, excipients and diluents which can be added to the edible or cosmetic composition of the present invention are approved for human and animal consumption or for cosmetic use, respectively.

According to some embodiments, the excipient may be suitable for either edible or cosmetic products. Non-limiting examples of such excipients may be selected from the group consisting of starch, maltodextrin, glucose, lactose, sucrose, gelatin, malt, glycol monostearate, sodium chloride, dried skim milk, glycerol, propylene glycol and combination thereof. According to some embodiments, the one or more additional cannabinoid compound(s) are present in one or more extracts of a Cannabis plant.

According to some embodiments, the Cannabis plant extract is formed through contact with a suitable solvent or a combination of solvents. According to some embodiments, the solvent is selected from the group consisting of a polar solvent, a hydrocarbon solvent, carbon dioxide, and a combination thereof.

Suitable solvents include but not limited to water, ethanol, ethyl acetate, CO₂, methanol, acetone, and acetic acid. According to one embodiment, the solvent is ethanol. According to another embodiment, the extraction is by CO₂. In particular, the term “extract” refers to a liquid or semi-solid or resinous substance obtained through extraction from plants defined in the present application, i.e. extracts obtained from Cannabis plant e.g. Cannabis sativa, Cannabis indica, and Cannabis ruderalis. In some embodiments, the term refers to a mixture of liquid or semi-solid, resinous substances obtained through extraction from two or more different plants. In some embodiments, the term refers also to a compound purified from the extract. According to some embodiments, the term “extract” has the meaning of a mixture or combination of two or more extracts.

The term “Cannabis extract” as used herein refers to one or more plant extracts from the Cannabis plant. A Cannabis extract contains, in addition to one or more cannabinoids, one or more non-cannabinoid components which are co-extracted with the cannabinoids from the plant material. Their respective ranges in weight will vary according to the starting plant material and the extraction methodology used. Cannabinoid-containing plant extracts may be obtained by various means of extraction of Cannabis plant material. Such means include but are not limited to supercritical or subcritical extraction with CO₂, extraction with hot or cold gas and extraction with solvents. In some embodiments, the term refers to a mixture of liquid or semi-solid, resinous substances obtained through extraction from two or more different Cannabis species. In some embodiments, the term refers also to a compound purified from the extract. The term “Cannabis plant” as used herein, refers to plants of the genus Cannabis, including but not limited to Cannabis sativa, Cannabis indica, and Cannabis ruderalis. According to some embodiment, Cannabis plant is a CBD-rich strain of Cannabis plant or THC-rich strain of Cannabis plant. Each possibility represents a separate embodiment.

According to some embodiments, the Cannabis plant extract is obtained from a strain selected from the group consisting of Cannabis sativa, Cannabis indica, Cannabis ruderalis, a hybrid strain, and combinations thereof. According to further embodiments the Cannabis plant extract is obtained from a strain selected from the group consisting of a high-CBD strain, a high-THC strain, and a combination thereof. According to some embodiments, the Cannabis plant extract comprises at least one cannabinoid selected from the group consisting of cannabidiol (CBD), tetrahydrocannabinol (THC), cannabinol (CBN), cannabigerol (CBG), cannabichromene (CBC), acids thereof and combinations thereof.

According to some embodiments, the Cannabis plant extract comprises from about 1 to about 25% (w/w) of CBD. According to some embodiments, the Cannabis plant extract comprises from about 1 to about 45% (w/w) of CBD. According to some embodiments, the Cannabis plant extract comprises about 1% (w/w) of CBD. According to some embodiments, the Cannabis plant extract comprises about 10% (w/w) of CBD. According to some embodiments, the Cannabis plant extract comprises about 25% (w/w) of CBD. According to some embodiments, the Cannabis plant extract comprises about 45% (w/w) of CBD.

According to some embodiments, the Cannabis plant extract comprises from about 1 to about 25% (w/w) of THC. According to some embodiments, the Cannabis plant extract comprises from about 1 to about 45% (w/w) of THC. According to some embodiments, the Cannabis plant extract comprises about 1% (w/w) of THC. According to some embodiments, the Cannabis plant extract comprises about 10% (w/w) of THC. According to some embodiments, the Cannabis plant extract comprises about 25% (w/w) of THC. According to some embodiments, the Cannabis plant extract comprises about 45% (w/w) of THC.

The term “hybrid strain” refers to different strains of Cannabis which include differing amounts and/or ratios of the various cannabinoid compounds. For example, Cannabis sativa typically has a relatively high THC/CBD ratio. Conversely, Cannabis indica has a relative low THC/CBD ratio compared to Cannabis sativa (although the absolute amount of THC can be higher in Cannabis indica than in Cannabis sativa).

As used herein the terms “high-CBD strain” and “CBD-rich strain” refer to a strain of Cannabis plant which comprises CBD and optionally one or more additional cannabinoids, such as, for example, but not limited to: THC, CBN, and the like. According to some embodiments, CBD is the main component in the high-CBD strain.

As used herein the terms “high-THC strain” and “THC-rich strain” are directed to a strain of Cannabis plant which comprises THC and optionally one or more additional cannabinoids, such as, for example, but not limited to: CBD, CBN, and the like. According to some embodiments, THC is the main component in the high-THC strain.

In the Cannabis plant, the concentrations of neutral cannabinoids, including CBD, are much lower than their acid form precursors. The neutral cannabinoids are formed artificially by non-enzymatic decarboxylation during extraction step, for example CBD is formed from CBDA (Yamauchi et. Al., Chem. Pharm. Bull., 1967, 15(7), 1075-1076; Takeda et. al, Toxicol. Lett., 2012, 214(3), 314-319).

According to some embodiments, composition comprises less than from about 0.01 to about 20% (w/w) of the cannabinoid component. According to some embodiments, the composition comprises less than about 20% (w/w) of the cannabinoid component. According to additional embodiments, the composition comprises less than about 10% (w/w) of the cannabinoid component. According to some embodiments, the composition comprises less than about 8% (w/w) of the cannabinoid component. According to additional embodiments, the composition comprises less than about 7% (w/w) of the cannabinoid component. According to further embodiments, the composition comprises less than about 5% (w/w) of the cannabinoid component. According to yet further embodiments, the composition comprises less than about 2% (w/w) of the cannabinoid component. According to still further embodiments, the composition comprises less than about 1% (w/w) of the cannabinoid component. According to additional embodiments, the composition comprises less than about 0.1% (w/w) of the cannabinoid component. According to additional embodiments, the composition comprises less than about 0.01% (w/w) of the cannabinoid component.

The cannabinoid component combination of the present invention is generally prepared by conventional methods such as are known in the art of making a mixture in the ratio described above. Such methods typically involve mixing of the CBDA ester and one or more additional cannabinoid compounds, or one or more extracts of a Cannabis plant in one or more steps to a relatively uniform state, with or without heating, cooling, application of vacuum, and the like.

### Cosmetic Compositions

According to some embodiments, the composition is a cosmetic composition selected from the group consisting of a make-up product, a foundation product, and a skin-care product. As used herein, the term “cosmetics” includes but is not limited to make-up, foundation, and skin care products. The term “make-up” refers to products that leave color on the face, including foundation, blacks and browns, i.e., mascara, concealers, eye liners, brow colors, eye shadows, blushers, lip colors, powders, solid emulsion compact, and so forth. The term “foundation” refers to liquid, cream, mousse, compact, and concealer or like product created or reintroduced by cosmetic companies to even out the overall coloring of the skin. Foundation is manufactured to work better over moisturized and/or oiled skin. As used herein, “excess moisture” means an undesirable and/or unhealthy level of bodily fluids deposited on human skin. “Skin care products” are those used to treat or care for, or somehow moisturize, improve, or clean the skin. Products contemplated by the phrase “skin care products” include, but are not limited to, adhesives, bandages, anhydrous occlusive moisturizers, antiperspirants, deodorants, personal cleansing products, nail polish, powders, tissues, wipes, hair conditioners-anhydrous, shaving creams and the like.

According to various embodiments the cannabinoid component can be emulsified, dissolved, dispersed or encapsulated in formulations suitable for use in either aqueous based or oil-based carriers.

The term “cosmetically acceptable carrier, excipient or diluent” is intended to mean, within the scope of a valid medical judgement, a carrier, excipient or diluent suitable for use in contact with the cells of humans and animals without toxicity, irritation, undue allergic response, and the like. It is appreciated that all carriers, excipients and diluents which can be added to the cosmetic composition of the present invention are approved for human and animal for cosmetic use.

Suitable carriers, excipients, or diluents include, but are not limited to, starch, glucose, lactose, sucrose, gelatin, silica dioxide, sodium stearate, glycol monostearate, talc, sodium chloride, dried skim milk, glycerol, propylene glycol, and waxes. Suitable waxes include but not limited to beeswax, lanolin, candelilla, soy, and carnauba wax.

Suitable further excipients include, but are not limited to, viscosity modifiers, emulsifiers, film-forming agents, foaming agents, colorants, preservatives, fragrance agents, solvents, electrolytes, pH adjusting agents, and combinations thereof.

Suitable viscosity modifier includes, but is not limited to, carboxymethyl cellulose, methyl cellulose, ethyl cellulose, hydroxyethyl cellulose, hydroxypropyl cellulose, hydroxypropylmethyl cellulose, silicates, and combinations thereof.

Suitable emulsifier includes, but is not limited to, lecithin, hydrogenated lecithin, glycerol, hydrogenated castor oil, cetyl alcohol, cetearyl alcohol, behenyl alcohol, butylene glycol, propylene glycol, and combinations thereof.

Suitable colorant includes, but is not limited to, inorganic pigments, organic pigments, and combinations thereof.

Suitable inorganic pigment includes, but is not limited to, rutile titanium dioxide, anatase titanium dioxide, zinc oxide, zirconium oxide, iron oxides, chromium oxide, chromium hydroxide, bismuth oxy chloride, manganese violet, cerium oxide, ultramarine blue, carmine, and combinations thereof.

Suitable organic pigment includes, but is not limited to, dihydroxyacetone (DHA), erythrulose, carbonyl derivatives, and combinations thereof.

Suitable preservative includes, but is not limited to, butyl paraben, methyl paraben, propyl paraben, propionate salts, quaternary ammonium compounds, butyl benzoate, benzyl alcohol, benzoic acid, and combinations thereof.

Suitable fragrance agent includes, but is not limited to, an essential oil obtained from a plant extract, a synthetic fragrance, and combinations thereof.

Suitable solvent includes, but is not limited to, water, ethanol, methanol, propanol, hexanol, propylene glycol, pentylene glycol, hexylene glycol, 1,2-octanediol, sorbitol, glycerol, ethyl acetate, and combinations thereof.

Suitable electrolyte includes, but is not limited to, sodium chloride.

Suitable pH adjusting agent includes, but is not limited to, organic and mineral acids or bases.

According to some embodiments, the cosmetic composition further comprises moisturizer. Suitable moisturizers include, but are not limited to, glycerin, hydrocarbon oils and waxes, including mineral oil, petrolatum, paraffin, ceresin, ozokerite, microcrystalline wax, polyethylene, and perhydrosqualene; silicone oils; triglyceride fats and oils, including those derived from vegetable, animal and marine source including jojoba oil and shea butter.

According to some embodiments, the cosmetic composition further comprises a cosmetically-active ingredient selected from the group consisting of sunscreen agents, anti-wrinkle and anti-aging agents, skin peel agents, whitening and bleaching agents, sunless tanning agents, vitamins, skin conditioning agents, and combinations thereof. Each possibility represents a separate embodiment of the invention.

Further suitable active agents and adjuvants used in compositions of the present invention are tabulated in The International Cosmetic Ingredient Dictionary and Handbook (INCI, 16th Edition, 2016). Generally, reference to specific materials utilizes the INCI adopted name nomenclature. The active agents and adjuvants are incorporated in the cosmetic compositions of the present invention in amounts that provide their intended functions, as those skilled in the cosmetic arts are knowledgeable. Generally, this amount is from about 0.001 to about 25%, more usually 0.01 to 15%, and in some specific cases 0.1 to 10% by weight of the composition.

Suitable sunscreen includes, but is not limited to, amino benzoic acid and derivatives, benzophenones, camphor derivatives, dibenzoyl methanes, salicylates, metal oxides, and combinations thereof.

Suitable anti-wrinkle and anti-aging agents include, but are not limited to, alpha hydroxy acids, beta hydroxy acids, fruit acids, sugar cane extract, glycomer, and combinations thereof.

Suitable skin peel agents include, but are not limited to, salicylic acid, lactic acid, glycolic, acetic acid and combinations thereof.

Suitable whitening and bleaching agents include, but are not limited to, hydroquinone and its derivatives, ascorbyl acid and its derivatives, and combinations thereof.

According to additional embodiments, the composition may further comprise vitamins. Suitable vitamins include e.g. vitamin A and derivatives, including retinoic acid, retinyl aldehyde, tretinoin (Retin A®), retinyl palmitate, adapalene, and beta-carotene; vitamin B (panthenol, provitamin B5, panthenic acid, vitamin B complex factor); vitamin C (ascorbic acid and salts thereof) and derivatives such as ascorbyl palmitate; vitamin D including calcipotriene (a vitamin D3 analog) vitamin E including its individual constituents alpha-, beta-, gamma-, delta-tocopherol and cotrienols and mixtures thereof and vitamin E derivatives including vitamin E palmitate, vitamin F linolate and vitamin E acetate; vitamin K and derivatives; vitamin Q (ubiquinone) and combinations thereof.

Suitable skin-conditioning agents include, but are not limited to, tocopherol and tocopherol derivatives, paraffin, xylitol, lactic acid, and combinations thereof. Skin-conditioning agents may be incorporated into this composition at 0.01 to 30% by weight of the total composition.

According to some embodiments, the amount of the CBDA ester varies from about 0.0001 to about 20% (w/w) of the total amount of the composition. According to some embodiments, the composition comprises less than about 20% (w/w) of the CBDA ester. According to additional embodiments, the composition comprises less than about 10% (w/w) of the CBDA ester. According to further embodiments, the composition comprises less than about 5% (w/w) of the CBDA ester. According to yet further embodiments, the composition comprises less than about 2% (w/w) of the CBDA ester. According to still further embodiments, the composition comprises less than about 1% (w/w) of the CBDA ester. According to additional embodiments, the composition comprises less than about 0.5% (w/w) of the CBDA ester. According to additional embodiments, the composition comprises less than about 0.1% (w/w) of the CBDA ester. According to additional embodiments, the composition comprises less than about 0.01% (w/w) of the CBDA ester.

The compositions may take on various forms including powder, cake, pencil, stick, ointment, cream, milk, lotion, liquid-phase, gel, emulsion, emulsified gel, mousse, foam, spray, wipes, liquid and powder foundation. The composition may be incorporated in cosmetically acceptable carriers or vehicles, such as but not limited to, liquid (e.g. suspension or solution), gel, emulsion, emulsified gel, mousse, cream, ointment, paste, serum, milk, foam, balm, aerosol, liposomes, solid (e.g. pressed powders), anhydrous oil and wax composition.

Alternatively, the composition is used in a liquid or powder foundations. More specifically, the compositions include facial skin care cosmetics such as skin lotion, skin milk, skin cream, gel, and make-ups such as foundation, foundation primer base, blush, lip stick, eye shadow, eye liner, nail enamel, concealed, mascara, and body make-up products.

Methods of Use of Cosmetic Compositions

In some embodiments, the cosmetic compositions are for use for improving appearance, well-being and/or general health. In addition, said compositions are suitable for use as a hair cosmetic, facial lines and skin imperfections. The methods of use for the compositions disclosed and claimed herein concern the improvement in the aesthetic appearance of skin and include, but are not limited to: methods of masking one or more of wrinkles, fine lines, pores, skin imperfections, especially in the facial, neck or on or around the lip areas; methods to correct imperfections in skin such as botches, freckles, redness, spider veins, and dark rings around the eyes: methods of enhancing or modifying skin color, methods to improve finished makeup, and methods for application to the hair, eyelashes, and eyebrows. According to some embodiments, the present invention provides a method for improving the appearance of skin comprising applying topically the composition to the skin of a subject. The term “subject” designates a mammal, optionally a human, but may also designate an animal including livestock or domestic animals (e.g., bovine, ovine, porcine, etc.) or those used for recreational purposes or as pets (e.g. dogs, cats, horses, etc.). According to some embodiments, the composition stimulates dermal production of collagen, elastin or Inhibitors of Metalloproteinases (TIMPs) and inhibits production of Matrix Metalloproteinases (MMPs).

Without wishing to be bound by theory or mechanism of action, it is contemplated that the CBDA esters act as precursors of the biological active natural cannabinoids, such as CBD and CBDA for cosmetic use. Without further wishing to being bound by theory or mechanism of action, the highly stable precursors of the present invention allow prolong biological activities of the stimulation of dermal production at least one of collagen, elastin or Tissue Inhibitors of Metalloproteinases (TIMPs), production of Matrix Metalloproteinases (MMPs), and of the anti-oxidative activity.

The term “precursor” refers to a precursor of a biologically active agent. Precursors must undergo a chemical or a metabolic conversion to become a biologically active agent. A precursor can be converted ex vivo to the biologically active agent by chemical transformative processes. In vivo, a precursor is converted to the biologically active agent by the action of a metabolic process, an enzymatic process or a degradative process that removes the precursor moiety to form the biologically active agent.

A person skilled in the art can select the appropriate presentation form, and also the method of preparing it, on the basis of general knowledge, taking into account the nature of the constituents used and the intended use of the composition.

The compositions described herein can be used by topically applying to the areas of the skin a safe and effective amount of the compositions. The effective amount can easily be determined by each user. As used herein the term, “safe and effective amount” refers to a sufficient amount of a compound, composition or other material described by this phrase to significantly induce a space filling of the appearance of the skin, but low enough to avoid undue side effects (e.g., significant skin irritation or sensitization), within the scope of sound judgment of the skilled person. The safe and effective amount of the compound, composition or other material may vary with the particular skin being treated, the age and physical condition of the biological subject being treated, the severity of the skin condition, the duration of treatment, the nature of concurrent therapy, the specific compound, composition, or other material employed, the particular cosmetically acceptable topical carrier utilized, and the factors within the knowledge and expertise of the skilled person.

Said composition can be applied once, twice or more times for several days, weeks, months or years at any intervals. The compositions are generally applied by light massaging the composition onto the skin. However, the method of application may be any method known in the art and is thus not limited to the aforementioned. Where necessary the compositions can be removed using soap and water or other cosmetic cleansers.

Methods of Preparation of Cosmetic Compositions

The compositions of the present invention are generally prepared by conventional methods such as are known in the art of making cosmetic compositions. Such methods typically involve mixing of the ingredients in one or more steps to a relatively uniform state, with or without heating, cooling, application of vacuum, and the like.

The compositions of the present invention may be formulated as single phase aqueous or non-aqueous compositions. According to some embodiments, the compositions described herein are formulated as emulsions. These emulsions may be oil-in-water (including silicone in water) emulsions, water-in-oil (including water-in-silicone) emulsions, or multiple emulsions such as oil-in-water-in-oil (o/w/o) or water-in-oil-in-water (w/o/w). It is understood that the oil phase can comprise silicone oils, non-silicone organic oils, or mixtures thereof. The compositions can comprise two immiscible phases that are admixed at the time of use by shaking. Each possibility represents a separate embodiment of the present invention.

In one embodiment, the composition is made by preparing a dispersion of each component in a suitably solvent (dispersant), adjusting the dispersion pH with a pH adjusting agent, and admixing the dispersions with shear to permit the formation of the desired matrix. In some instances, owing to the properties of the constituents it may be necessary to preheat one or both of the dispersants. The pH adjusting agent may also be provided into the admixed dispersions rather than into each dispersion individually. Certain of the adjuvants may require addition as premixes with a solvent, as generally known in the cosmetic art.

A person skilled in the art can select the appropriate presentation form, and also the method of preparing it on the basis of general knowledge, taking into account the nature of the constituents used and the intended use of the composition. Kits containing the above compositions are also contemplated. Compositions of the present invention can be packaged to contain, separately or in kit form together with a container, instructions or instruction brochure.

### Edible Compositions

According to some embodiments, the edible or potable composition is selected from the group consisting of food products, nutraceuticals, beverages, and animal feed.

As used herein, the term “edible” includes foodstuffs, nutraceuticals, beverage products, animal feed and drinking water additives. As used herein, the term “potable” includes beverage products and drinking water additives. The terms “edible” and “potable” refer to a material, component, composition, compound or formulation which is suitable and safe for eating and drinking As used herein, the term “nutraceutical additive” or “food additive” refers to a composition that may be consumed on its own as an edible or potable composition or may be added to foodstuffs, animal feedstuff, animal drinking water or beverages.

The term “edible carrier, excipient or diluent” is intended to mean, within the scope of a valid medical judgement, a carrier, excipient or diluent suitable and safe for consumption by humans and animals without toxicity, irritation, undue allergic response, and the like.

The edible composition may further comprise an edible excipient selected from starch, maltodextrin, glucose, lactose, sucrose, gelatin, malt, rice, flour, calcium carbonate, glycol monostearate, sodium chloride, dried skim milk, glycerol, propylene glycol and combinations thereof.

The composition may be incorporated in edible carriers or vehicles, such as but not limited to, liquid (e.g. suspension or solution), gel, emulsion, emulsified gel, mousse, cream, ointment, paste, serum, milk, foam, balm, aerosol, liposomes, solid (e.g. pressed powders), anhydrous oil and wax composition.

The edible carrier may be present in the edible composition in an amount of from about 0.5 to about 60% by weight of the edible composition. According to some embodiments, the physiologically acceptable carrier comprises starch. According to some embodiments, the starch is produced from wheat, potato, corn, rice, cassava (tapioca), or any combination thereof. The starch may be modified or unmodified and those skilled in the art will understand how to select from one or more starches that may be used in the present invention. Each possibility represents a separate embodiment of the present invention.

According to yet further embodiments, the edible composition comprises an edible excipient selected from the group consisting of fillers, emulsifiers, anticaking agents, preservatives, colorants, flavoring agents, and combinations thereof.

The nature of the CBDA ester of the present invention is hydrophobic. The hydrophobicity of molecule is defined by their octanol/water partition coefficient X log P. As used herein, the terms “hydrophobic” and “substantially water-insoluble” refer to a molecule/compound/material/component having an octanol/water partition coefficient X log P greater than 2, in certain embodiments greater than 3, and in other embodiments greater than 4.

Amphiphilic materials such as phospholipids and emulsifiers are materials that have both hydrophobic and hydrophilic domains that enable hydrophobic molecules to be solubilized in water by forming micelles and bilayers Amphiphilic materials prevent phase separation of the hydrophobic molecules by maintaining the hydrophobic groups in water through formation of strong hydrogen bonds with water molecules.

According to some embodiments, the CBDA ester is consumed in an aqueous solution. According to some embodiments, the edible composition further comprises an emulsifier or a phospholipid, wherein enabling homogeneous dispersion of the CBDA ester in the aqueous solution.

According to some embodiments, the edible composition further comprises phospholipids. According to some embodiments, the phospholipid is selected from the group consisting of naturally occurring phospholipids and synthetic phospholipids. According to further embodiments, the naturally occurring phospholipid is selected from the group consisting of soy lecithin, egg lecithin, hydrogenated soy lecithin, hydrogenated egg lecithin, and a combination thereof. According to some embodiments, the synthetic phospholipid is selected from the group consisting of phosphocholines, phosphoethanolamines, phosphatidic acids, phosphoglycerols, phosphoserines, mixed chain phospholipids, lysophospholipids, pegylated phospholipids, and a combination thereof. According to various embodiments the phospholipids may form micelles, emulsions or liposomes. According to some embodiments, the edible composition further comprises cholesterol.

According to additional embodiments, the filler is selected from the group consisting of starch, maltodextrin, dextrin, and sugars such as glucose, lactose and sucrose, and combinations thereof. Filler may be present in the edible composition in an amount of from about 0.5 to about 60% by weight of the edible composition. Each possibility represents a separate embodiment of the present invention.

Suitable emulsifier includes, but is not limited to hydrogenated lecithin, sodium phosphates, mono- and diglycerides, sodium stearoyl lactylate, diacetyl tartaric acid ester of monoglyceride, and combinations thereof.

Emulsifiers may be present in the edible composition in an amount of from about 0.5 to about 40% by weight of the edible composition.

Suitable anticaking agent includes, but is not limited to, cellulose, microcrystalline cellulose, silicon dioxide, tri-calcium phosphate, sodium chloride, sodium bicarbonate, sodium aluminum silicate, magnesium stearate, and magnesium carbonate. Each possibility represents a separate embodiment of the present invention. Anticaking agent may be present in the edible composition in an amount of from about 0.5 to about 10% by weight of the edible composition.

According to some embodiments, the preservative is selected from the group consisting of sulfur dioxide and sulfites, sorbic acid, sodium sorbate, calcium sorbate, potassium sorbate, benzoic acid, sodium benzoate, potassium benzoate, lactic acid, propionic acid, sodium propionate, and combinations thereof. Each possibility represents a separate embodiment of the present invention. Preservative may be present in the edible composition in an amount of from about 0.5 to about 10% by weight of the edible composition.

According to some embodiments, the colorant is selected from the group consisting of brilliant blue (E133), indigotin (E132), fast green (E143), erythrosine (E127), allura red (E129), tartrazine (E102), sunset yellow (E110), and natural food dyes.

According to additional embodiments, the natural food dye is selected from the group consisting of annatto (E160b), caramel (E150a-d), carmine (E120), dactylopius coccus, elderberry juice (E163), lycopene (E160d), paprika (E160c), turmeric (E100), and combinations thereof. Colorant may be present in the edible composition in an amount of from about 0.5 to about 10% by weight of the edible composition. Each possibility represents a separate embodiment of the present invention.

According to further embodiments, the flavoring agent is extracted from apple, cherry, green tea, cinnamon, clove, black tea, plum, mango, date, watermelon, coconut, pear, jasmine, peach, fennel, lychee, mint, chocolate, coffee, cream, banana, almond, grape, strawberry, blueberry, blackberry, pine, kiwi, sapote, taro, lotus, pineapple, orange, lemon, melon, licorice, vanilla, rose, osmanthus, ginseng, spearmint, citrus, cucumber, honeydew, walnut, honey, or combinations thereof. Each possibility represents a separate embodiment of the present invention. Flavoring agent may be present in the edible composition in an amount of from about 0.5 to about 10% by weight of the edible composition.

According to some embodiments, the edible or potable composition may further comprise water.

According to some embodiments, the edible composition may further comprise a pH adjusting agent to provide the desired pH of the edible composition. Suitable pH adjusting agents include e.g. organic and mineral acids or bases as are well known in the edible arts. Buffers to maintain the established pH may also be incorporated, for example sodium lactate.

The edible compositions of the present invention may further include nutrients. The terms “nutrient” as used herein refer to a compound having essential nutrition value. The amount of the nutrient will depend on the desired effect and the nutrient that is selected. In general, the amount of nutrient varies from about 0.0001 to about 50% (w/w). Alternatively, however the amount of nutrient in the final composition will vary from about 0.01 to about 20% (w/w) and alternatively from about 0.1 to about 10% (w/w). The nutrient may be formulated into the edible or potable composition of the present invention in any desired manner (e.g. mixed, stirred) under any desired condition (e.g. heated; under pressure) and in any desired form (e.g. a solid, liquid, gel, crystal, suspension).

According to some embodiments, the edible or potable composition may further comprise one or more edible nutrients that may be used in the context of the present invention. Suitable nutrients include, but are not limited to, vitamins, minerals, and combinations thereof.

According to additional embodiments, the vitamin is selected from the group consisting of vitamin A (retinols and carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), vitamin K (quinones), and combinations thereof. Each possibility represents a separate embodiment of the present invention.

According to further embodiments, the mineral is selected from the group consisting of magnesium, calcium, iron, zinc, chrome, selenium, potassium, silicon, and combinations thereof. Each possibility represents a separate embodiment of the present invention.

According to some embodiments, the edible composition is in a form selected from the group consisting of powder, flake, granular, capsule, tablet, syrup, solution, emulsion and suspension. In some embodiments the food product is selected from the group consisting of bread, cereals, pasta, pastry, biscuits, candy, and confectionary. In some embodiments the product comprising the edible compositions of the invention is a beverage. In some embodiments the beverage is selected from the group consisting of mineral water, aerated water, fruit beverage, fruit juice, syrup, beer, and alcoholic beverages.

The composition may be incorporated in edible vehicles, such as but not limited to, solid (e.g. powders), liquid (e.g. suspension or solution), gel, emulsion, emulsified gel, mousse, cream, paste, milk, and foam. Each possibility represents a separate embodiment of the present invention.

According to some embodiments, the amount of the CBDA ester varies from about 0.0001 to about 20% (w/w) of the total amount of the edible composition. According to further embodiments, the edible composition comprises less than about 20% (w/w) of the CBDA ester. According to additional embodiments, the edible composition comprises less than about 10% (w/w) of the CBDA ester. According to further embodiments, said composition comprises less than about 2% (w/w) of the CBDA ester. According to some embodiments, the edible composition comprises less than about 1% (w/w) of the CBDA ester. According to further embodiments, said composition comprises less than about 0.5% (w/w) of the CBDA ester. Each possibility represents a separate embodiment of the present invention.

Methods of Use of Edible Compositions

In some embodiments, the edible compositions of the present invention are directed for use as a foodstuff, animal feedstuff, animal drinking water and beverage products. A person skilled in the art can select the appropriate presentation form, and also the method of preparing it, on the basis of general knowledge, taking into account the nature of the constituents used and the intended use of the edible composition.

Without wishing to be bound by theory or mechanism of action, it is contemplated that the CBDA esters act as precursors of the biological active natural cannabinoids, such as CBD and CBDA for use in non-pharmaceutical application including foodstuffs, nutraceuticals, beverages and animal feed. Without further wishing to be bound by theory or mechanism of action, the highly stable precursors of the present invention allow prolonged beneficial effect on dysbiosis.

The term “precursor” refers to a precursor of a biologically active agent. Precursors must undergo a chemical or a metabolic conversion to become a biologically active agent. A precursor can be converted ex vivo to the biologically active agent by chemical transformative processes. In vivo, a precursor is converted to the biologically active agent by the action of a metabolic process, an enzymatic process or a degradative process that removes the precursor moiety to form the biologically active agent.

The compositions described herein can be used by adding or mixing together or alone combined in the daily nutrition. The effective amount can easily be determined by each user as used within the scope of sound judgment of the skilled person.

According to another aspect, the present invention provides a method for improving the physical well-being and/or the mental perception of well-being of a subject comprising consuming by said subject the edible composition of the present invention. According to some embodiments, the composition has a beneficial effect on the microbiome of the subject. According to additional embodiments, the improving of the physical well-being and/or the mental perception of well-being comprises beneficial effect on the microbiome of the subject. For this invention, the term “subject” designates a mammal, optionally a human, but may also designate an animal including livestock or domestic animals (e.g., bovine, ovine, porcine, etc.) or those used for recreational purposes or as pets (e.g. dogs, cats, horses, etc.).

The term “safe and effective amount” refers to a sufficient amount of a compound, composition or other material described by this phrase to significantly induce subject's physical well-being and/or the mental perception of well-being (e.g., heart health, strength of bone and teeth, higher energy levels, weight control, brain health, temperament, and emotional state). The safe and effective amount of the compound, composition or other material may vary with the physical well-being and/or the mental perception of well-being of the biological subject being treated, the duration of treatment, the nature of concurrent therapy, the specific compound, composition, or other material employed, the particular edible or potable carrier utilized, and the factors within the knowledge and expertise of the skilled person.

Said compositions can be consumed once, twice or more times for several days, weeks, months or years at any intervals. The edible or potable compositions are generally consumed by oral administration.

Methods of Preparation of Edible Compositions

The edible compositions of the present invention are generally prepared by conventional methods such as are known in the art of making edible compositions. Such methods typically involve mixing of the ingredients in one or more steps to a relatively homogenized mixture, with or without heating, cooling, application of vacuum, and the like.

The edible compositions of the present invention may be formulated as non-aqueous or as single-phase aqueous compositions. According to some embodiments, the edible composition is in the form of powder. These powders may be mix or add at the time of use by shaking.

According to some embodiments, the edible or potable composition is made by preparing a dispersion of each component in a suitably solvent (dispersant), adjusting the dispersion pH with a pH adjusting agent, and admixing the dispersions with shear to permit the formation of the desired mixture. In some instances, owing to the properties of the constituents it may be necessary to preheat one or both of the dispersants. The pH adjusting agent may also be provided into the admixed dispersions rather than into each dispersion individually. Certain of the excipients may require addition as premixes with a solvent, as generally known in the art. Each possibility represents a separate embodiment of the present invention.

It is appreciated that all carriers, excipients, and diluents used in the present invention are generally recognized as safe (GRAS) approved for human and animal consumption.

The cannabinoid component combination of the present invention is generally prepared by conventional methods such as are known in the art of making a mixture in the ratio described above. Such methods typically involve mixing of the cannabidiolic acid ester and one or more additional cannabinoid compounds in one or more steps to a relatively uniform state, with or without heating, cooling, application of vacuum, and the like.

A person skilled in the art can select the appropriate presentation form, and also the method of preparing it on the basis of general knowledge, taking into account the nature of the constituents used and the intended use of the composition.

The following examples describe specific aspects of the invention to illustrate the invention and provide a description of the present methods for those skilled in the art. It should be noted that the term “and” or the term “or” is generally employed in its sense including “and/or” unless the context clearly dictates otherwise. As used herein, the term “about” is meant to encompass variations of ±10%.

### EXAMPLES

### Example 1

### Synthesis of Cannabidiolic Acid (CBDA)

The preparation process described in the PCT publication WO 2018/235079 was applied. A mixture of Cannabidiol (CBD, 314 mg, 1 mmol) and 2 molar solution of Magnesium Methyl Carbonate (MMC/2M, 1.5 ml, 3 mmol) in dimethylformamide (DMF) was heated at 130° C. for 3 hours. Then the reaction was cooled to 0° C., acidified with 10% hydrochloric acid and extracted with ether. The organic layer was washed with saline, dried over the drying agent magnesium sulfate (MgSO₄) and then evaporated. The crude compound was then cleaned by column chromatography (20% ether-petroleum ether).

### Example 2

### Synthesis of Cannabidiolic Acid Methyl Ester (HU-580)

The preparation process described in the PCT publication WO 2018/235079 was applied. To a solution of Cannabidiolic Acid (CBDA) (175 mg, 0.488 mmol) in 2.5 ml dichloromethane (CH₂Cl₂), was added 0.02 ml of methanol (CH₃OH, 0.488 mmol) and 7.2 mg of 4-Pyrrolidinopyridine (0.048 mmol). The reaction was stirred for 5 minutes at room temperature followed by the addition of the coupling agent, N,N′ Dicyclohexylcarbodiimide (DCC) (121 mg, 0.585 mmol) and stirred overnight. Then the solvent was evaporated and the crude mixture acidified with 5% hydrochloric acid and extracted with dichloromethane (CH₂Cl₂). The organic layer was washed with saturated aqueous sodium bicarbonate (NaHCO₃), dried over the drying agent magnesium sulfate (MgSO₄) and then evaporated. The crude compound is then cleaned by column chromatography (2% ether-petroleum ether).

¹H-NMR spectra were obtained using a Bruker AMX 300 MHz apparatus using the deuterated DMSO. Thin-layer chromatography (TLC) was run on silica gel 60F254 plates (Merck). Column chromatography was performed on silica gel 60 Å(Merck). Compounds were located using a UV lamp at 254 nm.

¹H NMR (300 MHz, ((CD₃)₂SO)) δ 6.18 (1H, s, Ar), 5.07 (1H, s), 4.44 (1H, s), 4.41 (1H, s), 3.82 (3H, s), 3.35 (1H, m), 2.66 (1H, m), 2.49 (2H, t), 2.09 (1H, b), 1.95 (3H, s),1.71-1.05 (12, ms), 0.86 (3H, t).

### Example 3

### Cannabinoid Component

The cannabinoid component of the present invention is generally prepared by conventional methods such as are known in the art of making a mixture, wherein the cannabinoid component comprises a cannabidiolic acid ester alone or in combination with one or more additional cannabinoid compound(s). Such methods typically involve mixing of the cannabidiolic acid ester and one or more cannabinoid compound(s) in one or more steps to a relatively uniform state, with or without heating, cooling, application of vacuum, and the like.

### Example 4

### Lip Balm

| Ingredient | Percentage (wt/wt) |
|---|---|
| Beeswax | 21.00 |
| Lanolin | 18.00 |
| Carnauba wax | 22.00 |
| Cocoa butter | 25.00 |
| Seed oil | 5.00 |
| Cannabinoid component | 3.00 |
| Tocopherol | 1.00 |
| Fragrance agent | 5.00 |
| Total | 100.00 |

In the first beaker a mixture of beeswax, lanolin and carnauba wax is heated and mixed to 65° C. and then is cooled to around 35° C. In the second beaker a mixture of cocoa butter, seed oil, cannabinoid component and tocopherol is heated and mixed to 40° C., then the mixture is poured to the first beaker, and then fragrance oil is added and mixed into the mixture. Finally, the resulted mixture is poured into cylindrical molds and is cooled down to room temperature.

### Example 5

### Efficacy of Reparative Leave-On Hair Serum

Placebo and cannabinoid activated leave-on reparative hair serums are prepared comprising the following: glycerin, water, starch, sucrose, alcohol, hydrogenated castor oil, a cannabinoid component (activated serum only), and additional excipients as seen fit. Samples of untreated, bleached, and bleached treated hair are used in the study. Bleached samples are damaged with a bleaching procedure based on an oxidizing agent whereby melanin and other hair components are oxidized. Following multiple rounds of no treatment, treatment with placebo serum, or treatment with activated serum, hair samples are evaluated for shine, strength, sorption, and morphology chances according to Benaiges et al. (Journal of Applied Polymer Sci., 2013, 861-868). Data is analyzed using a three-factor ANOVA for effect on shine, strength, sorption and morphology changes (p<0.05).

### Example 6

### Efficacy of Anti-Aging Cream

Anti-aging creams serve to reduce, mask or prevent signs of skin aging. An anti-aging cream is prepared using methods known in the art, comprising a cannabinoid component, stearic acid, cetyl alcohol, liquid paraffin, glycerin, methyl paraben, propylene glycol, and distilled water. Evidence of anti-aging effects is examined via self-assessment and clinical assessment using the following parameters: wrinkles and roughness, skin hydration, skin elasticity, and skin color. The cream is tested for cytotoxicity, skin irritation, and allergic sensitization according to Nigam (Indian J. Dermatol. Venereol. Leprol., 2009, 10-19).

### Example 7

### Moisturizing and Exfoliating Cuticle Cream

Cuticle cream is designed to moisturize, exfoliate and address a variety of cosmetic conditions of the skin in the fingernail area, for example, dry skin or hangnails. Cuticle cream works to exfoliate and condition living skin as well as remove dead skin from the nail plate. A cuticle cream is made using methods known in the art comprising a cannabinoid component, stearic acid, glycerin, propylene glycol, lanolin, beeswax, cetyl alcohol, mineral oil, fragrance, colorants, and water. The cream is tested for cytotoxicity, skin irritation, and allergic sensitization according to Nigam (Indian J. Dermatol. Venereol. Leprol., 2009, 10-19).

### Example 8

### Powdered Edible Additive

A powdered additive is formulated by blending filler such as tapioca maltodextrin or microcrystalline cellulose or a mixture thereof, emulsifier such as soy lecithin granules, CBDA-ME, and flavoring agent. All ingredients, as detailed above, are added in a high-speed shearing device at room temperature.

### Example 9

### Formulations of Drinking-Water Additive

A drinking-water additive is formulated by forming liposomes that are loaded with CBDA-ME, and then dispersed in water. Optionally an approved food acceptable organic solvent such as ethanol or PEG is added.

### Example 10

### Cannabinoid Coated Powdered Sugar

Powdered sugar is added to a polypropylene container with rounded corners and an impression is made into the center. Warm liquid cannabinoids are poured into the impression so as to not touch the sides of the container. A lid is screwed onto the container and the container placed in a refrigerator to harden the cannabinoids. The chilled container is placed in the Dual Asymmetrical Centrifuge (DAC) and run as needed to achieve visual homogeny. If a blob of cannabinoids still remains the container is run for additional intervals until the powder is homogeneous.

### Example 11

### Cannabinoid Enriched Oil

Cannabinoid component is dissolved in vegetable oil, heating as necessary, for a specified time period. Oil is used in edible products for human or animal consumption. Oil is incorporated at a desired stage during the production of an edible product. Oil may be sprayed on an edible product during a final “enriching phase” alongside materials including, but not limited to, preservatives and flavoring agents.

### Example 12

### Pet Food

The ingredients of pet food may comprise, but are not limited to, byproducts of meat, poultry, and seafood, feed grains, soybean meal, corn meal, cracked wheat, barley, water, meat broth, blood, salt, preservatives, stabilizers, gelling agents, bean and guar gums, cellulose, carrageenan, and other starches.

Dry Food:

Ingredients are brought together in a mixer and formed into a moist dough. The dough is cooked under intense heat and pressure, then cut and shaped into “kibble” which rapidly is expand when exposed to standard air pressure. Kibble is dried in an oven, cooled, and sprayed with a flavor enhancer and a cannabinoid component.

Wet Food:

Ingredients, including a cannabinoid component, are incorporated in a mixer. Empty containers are filled, sealed, and cooked at a specified temperature for a desired time to destroy contaminants.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A cosmetic or edible composition, comprising:
a cannabinoid component, wherein the cannabinoid component comprises a cannabidiolic acid (CBDA) ester represented by the structure of Formula (I), alone or in combination with one or more additional cannabinoid compound(s), and a physiologically acceptable carrier, excipient or diluent, 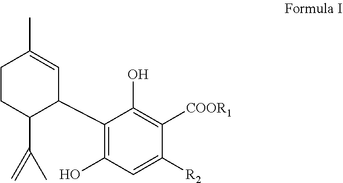
wherein:
R₁ and R₂ are each independently selected from the group consisting of a linear or branched C₁-C₁₀ alkyl, a linear or branched C₂-C₁₀ alkenyl, and a linear or branched C₂-C₁₀ alkynyl;
and stereoisomers and salts thereof;
and with the proviso that when the composition comprises a CBDA ester of formula (I) wherein R₁ is methyl and R₂ is a C₃ or C₅ linear alkyl, the CBDA ester comprises more than 50% of the total weight of the cannabinoids in the composition.

2. The composition according to claim 1, wherein R₂ is other than a C₅ linear alkyl.

3. The composition according to claim 1, wherein R₂ is other than a C₃ linear alkyl.

4. The composition according to claim 1, wherein R₁ is methyl.

5. The composition according to claim 4, wherein the cannabidiolic acid ester is cannabidiolic acid methyl ester (CBDA-ME).

6. The composition according to claim 1, further comprising one or more additional cannabinoid compound selected from the group consisting of cannabidiol (CBD), cannabigerol (CBG), Δ⁸-tetrahydrocannabinol (Δ⁸-THC), Δ⁹-tetrahydrocannabinol (Δ⁹-THC), cannabinol (CBN), Δ⁹(11)-tetrahydrocannabinol (exo-THC), cannabichromene (CBC), tetrahydrocannabinol-C3 (THC-C3), tetrahydrocannabinol-C4 (THC-C4), tetrahydrocannabinol-C7 (THC-C7), esters thereof and combinations thereof.

7. (canceled)

8. (canceled)

9. (canceled)

10. (canceled)

11. (canceled)

12. (canceled)

13. (canceled)

14. (canceled)

15. The composition according to claim 1, comprises from 0.01% to about 8% (w/w) of the cannabinoid component.

16. The composition according to claim 1, wherein the cannabinoid component is emulsified, dissolved, dispersed or encapsulated in formulations suitable for use in either aqueous based or oil-based carriers.

17. The composition according to claim 1, wherein said composition is a cosmetic composition selected from the group consisting of a make-up product, a foundation product, and a skin-care product.

18. (canceled)

19. The composition according to claim 1, wherein the physiologically acceptable excipient is selected from the group consisting of viscosity modifiers, emulsifiers, film-forming agents, foaming agents, colorants, preservatives, fragrance agents, solvents, electrolytes, pH adjusting agents, and combinations thereof.

20. The composition according to claim 1, further comprising a cosmetically-active ingredient selected from the group consisting of sunscreen agents, anti-wrinkle agents, anti-aging agents, skin peel agents, bleaching agents, sunless tanning agents, vitamins, skin conditioning agents, and combinations thereof.

21. The composition according to claim 1, in a form selected from the group consisting of powder, ointment, cream, milk, lotion, gel, emulsion, emulsified gel, mousse, foam, spray, liquid, paste, serum, and aerosol.

22. A method for improving the appearance of skin comprising applying topically to the skin of a subject the cosmetic composition according to claim 1.

23. The composition according to claim 1, wherein said composition is an edible composition selected from the group consisting of food products, nutraceuticals, beverages, and animal feed.

24. (canceled)

25. The composition according to claim 23, further comprising a phospholipid selected from the group consisting of soy lecithin, egg lecithin, hydrogenated soy lecithin, hydrogenated egg lecithin, and a combination thereof.

26. The composition according to claim 25, in the form of a micelle, emulsion, or liposome.

27. (canceled)

28. The composition according to claim 25, further comprising cholesterol.

29. The composition according to claim 23, wherein the physiologically acceptable excipient is an edible excipient selected from the group consisting of fillers, emulsifiers, anticaking agents, preservatives, colorants, flavoring agents, and combinations thereof.

30. The composition according to claim 23, further comprises a nutrient selected from the group consisting of vitamins, minerals, and combinations thereof.

31. The composition according to claim 1, wherein said composition is in a form selected from the group consisting of powder, flake, granular, capsule, tablet, syrup, solution, emulsion and suspension.

32. (canceled)

33. (canceled)

34. (canceled)
